(19) United States
(12) Patent Application Publication
(10) Pub. No.: US 2025/0101046 A1
(43) Pub. Date: Mar. 27, 2025

(54) Synthesis for Borylated Amino Acid Compositions Comprising TC220 and TC221 For Use in Boron Neutron Capture Therapy and Methods Thereof

(71) Applicant: TAE Life Sciences LLC, Irvine, CA (US)

(72) Inventors: Tioga J. Martin, Los Angeles, CA (US); Michael Y. Torgov, Redondo Beach, CA (US); Paul Zhichkin, Slingerlands, NY (US)

(21) Appl. No.: 18/807,293

(22) Filed: Aug. 16, 2024

Related U.S. Application Data
(60) Provisional application No. 63/628,747, filed on Aug. 17, 2023.

(51) Int. Cl. C07F 5/02; A61K 41/00

(52) U.S. Cl. CPC C07F 5/025; A61K 41/0095

(57) ABSTRACT

Borylated Amino Acid compositions comprising tyrosine derivatives TC220 and TC221 and improved methods of making TC220 and TC221 are disclosed herein. Consequently, the TC220 and/or TC221 can be scaled up to commercial scale and administered to patients as a Neutron Capture Agent and provide a method of treating cancer, immunological disorders, and other disease by utilizing a Neutron Capture Therapy modality.

### CROSS-REFERENCE TO RELATED APPLICATIONS

This application claims priority to U.S. Provisional Patent Application No. 63/628,747 filed 17 Aug. 2023, the contents of which are fully incorporated by reference herein.

### STATEMENT OF RIGHTS TO INVENTIONS MADE UNDER FEDERALLY SPONSORED RESEARCH

Not applicable.

### FIELD OF THE INVENTION

The invention described herein relates to the field of boron neutron capture therapy (BNCT). Specifically, the invention relates to improved synthesis methods for borylated amino acid (“BAA”) or (“BAAs”) compositions, including but not limited to BAAs denoted TC220 and TC221, which can be used as a vehicle for neutron capture therapy in humans. The invention further relates to the treatment of cancers and other immunological disorders and diseases.

### BACKGROUND OF THE INVENTION

Cancer is the second leading cause of death next to coronary disease worldwide. Millions of people die from cancer every year and in the United States alone cancer kills well over a half-million people annually, with 1,688,780 new cancer cases diagnosed in 2017 (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. In particular, carcinomas of the lung (18.4% of all cancer deaths), breast (6.6% of all cancer deaths), colorectal (9.2% of all cancer deaths), liver (8.2% of all cancer deaths), and stomach (8.2% of all cancer deaths) represent major causes of cancer death for both sexes in all ages worldwide (GLOBOCAN 2018). These and virtually all other carcinomas share a common lethal feature in that they metastasize to sites distant from the primary tumor and with very few exceptions, metastatic disease fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Furthermore, many cancer patients experience a recurrence of their disease.

Although cancer therapy has improved over the past decades and survival rates have increased, the heterogeneity of cancer still demands new therapeutic strategies utilizing a plurality of treatment modalities. This is especially true in treating solid tumors at anatomical crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients' quality of life.

Neutron Capture Therapy (NCT) is a promising form of radiation therapy. It is a technique that selectively kills tumor cells using boron compound while sparing the normal cells. BNCT relies on the propensity of non-radioactive ¹⁰B isotope to absorb epithermal neutrons that fall into the low energy range of 0.5 keV<Eₙ<30 keV. Following neutron capture, boron atom undergoes a nuclear fission reaction giving rise to an alpha-particle and a recoiled lithium nucleus (7Li) as follows:

¹⁰B+n→⁷Li+⁴He

The alpha particle deposits high energy i.e., 150 keV/μm along their short path essentially restricted to a single cell diameter that results in a double strand DNA break followed by cancer cell death by apoptosis. Thus, BNCT integrates a concept of both chemotherapy, targeted therapy, and the gross anatomical localization of traditional radiotherapy.

Even though the conceptual techniques of NCT and specifically Boron Neutron Capture Therapy (BNCT) are well known, the technological limitations associated with this type of treatment have slowed progress. During the early investigations using the research reactors of MIT in 1960's, several dozens of patients were treated using disodium decahydrodecaborate, which was considered less toxic than simple boron compounds used previously yet capable of delivering more boron to the cell. Unfortunately, BNCT studies were halted in the USA due to the severe brain necrosis in the patients undergoing BNCT and the potential harm of using nuclear reactors.

Hiroshi Hatanaka in 1968 re-investigated clinical application of BNCT in Japan using sodium borocaptate (BSH) by directing the beam to surgically exposed intracranial tumor and reported of achieving 58% of 5-year survival rate. In 1987 clinicians in Japan applied BNCT for the treatment of malignant melanoma using boronophenylalanine (BPA) as boron compound. Thus, slow resurgence of BNCT took place albeit limited to the countries with an access to research reactor facilities capable of delivering epithermal neutron beams. Currently, given the technological improvements in both (i) the infusion and delivery of a capture compound, which preferably concentrates in the tumor, and (ii) more abundant and easier access to neutron beam using cyclotrons, there has been a resurgence in NCT treatment methods.

The proton boron fusion reaction relies on the naturally abundant 11B isotope rather than 10B required for BNCT. Unlike BNCT, three alpha particles are emitted after the fusion reaction between a proton (¹H) and a boron (¹¹B) nucleus: p+¹¹B->3α. The proton beam has the advantage of a Bragg-peak characteristic reducing the normal tissue damage and when combined with proton capture, may improve the efficacy of the proton therapy alone.

Carriers of boron have evolved since the 1950s and are reviewed in NEDUNCHEZHIAN, et. al., J. Clin. Diag. Res., vol. 10 (12) (December 2016). Briefly, the 1^{st} generations of boron compounds represented by boric acid and its derivatives were either toxic or suffered from low tumor accumulation/retention. BPA and BSH are both considered the 2^{nd} generation compounds that emerged in 1960s. These had significantly lower toxicity and better PK and biodistribution. BPA-fructose complex is considered the 3^{rd} generation compound that is used to treat patients with H&N, glioblastoma and melanoma using BNCT since 1994. BPA-fructose and BSH are the only compounds that are being used in clinic as boron carriers to date although both low and high molecular weight biomolecules such as nucleosides, porphyrins, liposomes, nanoparticles and mAbs have been evaluated for the tumor targeting in preclinical models. The main deficiency of BPA-fructose is relatively low solubility combined with its rapid clearance that prevents achieving high or peak serum dosage (Cₘₐₓ) in blood, one of the drivers influencing the tumor uptake.

From the aforementioned, it will be readily apparent to those skilled in the art that a new treatment paradigm is needed in the treatment of cancers and immunological diseases. By using modern chemical synthesis and modifying natural amino acids with boron, a new disease treatment can be achieved with the overall goal of more effective treatment, reduced side effects, and lower production costs.

Given the current deficiencies associated with NCT, it is an object of the present invention to provide new and improved methods of treating cancer(s), immunological disorders, and other diseases utilizing borylated amino acids and NCT.

### SUMMARY OF THE INVENTION

The invention provides for compositions comprising natural amino acids which have been borylated via chemical synthesis for use as a delivery modality to treat human diseases such as cancer, immunological disorders, including but not limited to rheumatoid arthritis, ankylosing spondylitis, and other cellular diseases, including but not limited to Alzheimer's disease. In certain embodiments, the borylated amino acids are comprised of naturally occurring amino acids such as phenylalanine, tryptophan, tyrosine, histidine, and any other naturally occurring amino acid set forth in Table I.

In a further embodiment, the invention comprises TC220.

In a further embodiment, the invention comprises TC221.

In a further embodiment, the invention comprises improved methods of synthesizing TC220.

In a further embodiment, the invention comprises improved methods of synthesizing TC221.

In a further embodiment, the invention comprises methods of concentrating Boron in a cell comprising (i) synthesizing a borylated amino acid (“BAA”); (ii) administering the BAA to a patient, and (iii) irradiating the cell with neutrons.

In a further embodiment, the invention comprises methods of concentrating Boron in a cell comprising (i) synthesizing TC220 (ii) administering the TC220 to a patient, and (iii) irradiating the cell with neutrons.

In a further embodiment, the invention comprises methods of concentrating Boron in a cell comprising (i) synthesizing TC221 (ii) administering the TC221 to a patient, and (iii) irradiating the cell with neutrons.

In another embodiment, the present disclosure teaches methods of synthesizing BAA's.

In another embodiment, the present disclosure teaches methods of treating cancer(s), immunological disorders, and other diseases in humans.

### BRIEF DESCRIPTION OF THE FIGURES

FIG. 1. Chemical Structure of TC220 Hydrochloride.

FIG. 2. Chemical Structure of TC221.

FIG. 3. Synthesis Schema for TC220 Hydrochloride.

FIG. 4. Preparation of(S)-2-(tert-Butoxycarbonylamino)-3-(4-hydroxy-3-iodophenyl) propionic acid.

FIG. 5. Preparation of trimethylsilyl(S)-2-(tert-butoxycarbonylamino)-3-[3-iodo-4-(trimethylsiloxy)phenyl]propionate.

FIG. 6. Preparation of ¹⁰B—(S)-2-(tert-Butoxycarbonylamino)-3-[3-(dihydroxyboryl)-4-hydroxyphenyl]propionic acid.

FIG. 7. Preparation of ¹⁰B-TC220 hydrochloride.

FIG. 8. Purity Profile of ¹⁰B-TC220 hydrochloride. FIG. 8A. Shows all analyzed wavelengths and mass spectrum TICs. FIG. 8B. Shows the positive M/Z of M+1=346.23. FIG. 8C. Shows the UV spectrum at 225 nm. FIG. 8D. Shows the area percent of detectable UV peaks.

FIG. 9. Synthesis Schema for Conversion of TC220 Hydrochloride to TC221.

FIG. 10. Synthesis Schema for Conversion of Compound 4 to Compound 15.

FIG. 11. Chemical Synthesis for N-Boc-Tyr (3-B(OH)₂, 4-OMe)-OMe to Boc-Tyr (3-B(OH)₂, 4-OMe)-OH.

FIG. 12. Chemical Synthesis for Boc-Tyr (3-B(OH)₂, 4-OMe)-OH to TC221.

### DETAILED DESCRIPTION OF THE INVENTION

### Outline of Sections

I.) Definitions
II.) BPA
III.) BSH
IV.) Boron
   a. Boron Generally
V.) Naturally Occurring Amino Acids
VI.) Borylated Amino Acids (BAAs)
   a. Amino Acid Compositions
   b. BAA Comprising Tyrosine (TC220 and TC221)
   c. New and Improved Synthesis of TC220 and TC221
VII.) Boron Neutron Capture Therapy Using TC220 & TC221
VIII.) Proton Boron Fusion Therapy Using TC220 & TC221
IX.) Methods of Delivering TC220 & TC221 to a Cell
X.) KITS/Articles of Manufacture

### I.) Definitions

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

The terms “advanced cancer”, “locally advanced cancer”, “advanced disease” and “locally advanced disease” mean cancers that have extended through the relevant tissue capsule and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease and these patients have substantially less favorable outcomes compared to patients having clinically localized (organ-confined) cancer.

“Amino Acid” means a simple organic compound containing both a carboxyl (—COOH) and an amino (—NH₂) group.

“Borylation” means reactions that produce an organoboron compound through functionalization of aliphatic and aromatic C—H bonds.

“Borylated Amino Acid” (BAA) means a compound comprising a naturally occurring amino acid, such as those set forth in Table I, which has undergone a borylation reaction. BAAs can be synthesized in multiple formats depending on the underlying amino acid that is being used.

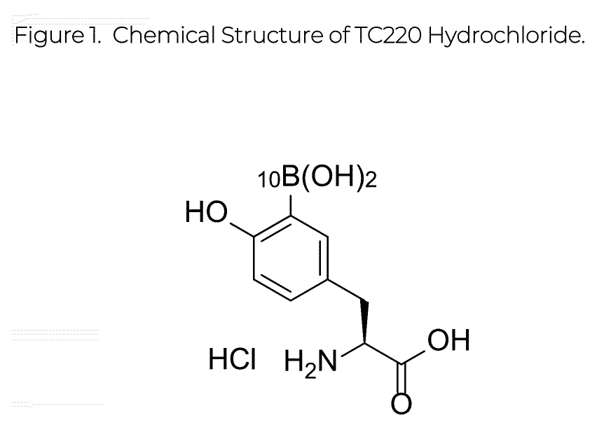

“TC220” means a compound comprising the chemical structure set forth in FIG. 1. For the purpose of this disclosure, TC220 may be denoted as “TC220” or “TC220 Hydrochloride”.

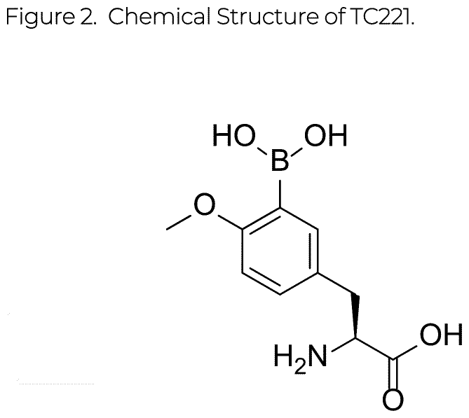

“TC221” means a compound comprising the chemical structure set forth in FIG. 2.

The term “compound” refers to and encompasses the chemical compound (e.g. a BAA) itself as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above

The terms “inhibit” or “inhibition of” as used herein means to reduce by a measurable amount, or to prevent entirely.

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “metastatic cancer” and “metastatic disease” mean cancers that have spread to regional lymph nodes or to distant sites and are meant to include stage D disease under the AUA system and stage T×N×M+ under the TNM system.

“Molecular recognition” means a chemical event in which a host molecule is able to form a complex with a second molecule (i.e., the guest). This process occurs through non-covalent chemical bonds, including but not limited to hydrogen bonding, hydrophobic interactions, and ionic interaction.

“Pharmaceutically acceptable” refers to a non-toxic, inert, and/or composition that is physiologically compatible with humans or other mammals.

The term “neutron capture agent” means a stable non-reactive chemical isotope which, when activated by neutrons produces alpha particles.

The term “neutron capture therapy” means a noninvasive therapeutic modality for treating locally invasive malignant tumors such as primary brain tumors and recurrent head and neck cancer and other immunological disorders and disease by irradiating a neutron capture agent with neutrons.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

### II.) BPA

By way of reference and for context of the prior art, (10B)-BPA, L-BPA, or 4-Borono-L-phenylalanine (Sigma Aldrich, St. Louis, MO) is a synthetic compound with the chemical formula C₉H₁₂BNO₄. The structure is shown below:

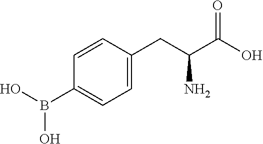

and is an important boronated compound useful in the treatment of cancer though BNCT. It is a widely known compound which many syntheses have been developed (See, U.S. Pat. No. 8,765,997, Taiwan Biotech Co, Ltd., Taoyuan Hsein, Taiwan, and US2017/0015684, Stella Pharma Corp., Osaka Prefecture Univ., Osaka, Japan).

### III.) BSH

In addition to BPA, BSH, or sodium borocaptate, or BSH sodium borocaptate, or Borocaptate sodium 10B, or undecahydro-closo-dodecaboratethiol is a known synthetic chemical compound with the chemical formula Na2B12H11SH. The structure is shown below:

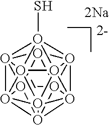

where boron atoms are represented by dots in the vertices for the icosahedron. BSH is used as a capture agent in BNCT. Generally speaking, BSH is injected into a vein and becomes concentrated in tumor cells. The patient then receives radiation treatment with atomic particles called neutrons. The neutrons fuse with the boron nuclei in BSH and to produce high energy alpha particles that kill the tumor cells.

### IV.) Boron

### (a.) Boron Generally

Generally speaking, and for purposes of this disclosure, Boron is a chemical element with symbol B and atomic number 5. Primarily used in chemical compounds, natural boron is composed of two stable isotopes, once of which is Boron-10 and the other is Boron-11. Boron-10 isotope is useful for capturing epithermal neutrons, which makes it a promising tool in a therapeutic context using Boron Neutron Capture Therapy. Biologically, the borylated compounds disclosed herein are nontoxic to humans and animals. Based on the foregoing, it will be readily apparent to one of skill in the art that improved modalities for providing high concentrations of boron into a cancer cell are advantageous. It is an object of the present disclosure to provide that advantage.

### V.) Naturally Occurring Amino Acids

Generally speaking, and for the purposes of this disclosure, naturally occurring amino acids are organic compounds containing amine (—NH₂) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids. About 500 naturally occurring amino acids are known (though only 20 appear in the genetic code (Table 1)) and can be classified in many ways. They can be classified according to the core structural functional groups' locations as alpha-(α-), beta-(β-), gamma-(γ-) or delta-(δ-) amino acids; other categories relate to polarity, pH level, and side chain group type (aliphatic, acyclic, aromatic, containing hydroxyl or sulfur, etc.). In the form of proteins, amino acid residues form the second-largest component (water is the largest) of human muscles and other tissues. Beyond their role as residues in proteins, amino acids participate in a number of processes such as neurotransmitter transport and biosynthesis.

The twenty (20) amino acids encoded directly by the genetic code (See, Table 1) can be divided into several groups based on their properties. Principal factors are charge, hydrophilicity or hydrophobicity, size, and functional groups. These properties are important for protein structure and protein-protein interactions. The water-soluble proteins tend to have their hydrophobic residues (Leu, lie, Val, Phe, and Trp) buried in the middle of the protein, whereas hydrophilic side chains are exposed to the aqueous solvent.

The integral membrane proteins tend to have outer rings of exposed hydrophobic amino acids that anchor them into the lipid bilayer. In the case part-way between these two extremes, some peripheral membrane proteins have a patch of hydrophobic amino acids on their surface that locks onto the membrane. In similar fashion, proteins that have to bind to positively charged molecules have surfaces rich with negatively charged amino acids like glutamate and aspartate, while proteins binding to negatively charged molecules have surfaces rich with positively charged chains like lysine and arginine. There are different hydrophobicity scales of amino acid residues.

Some amino acids have special properties such as cysteine, which can form covalent disulfide bonds to other cysteine residues, proline that forms a cycle to the polypeptide backbone, and glycine that is more flexible than other amino acids.

### VI.) Borylated Amino Acids (BAA)

By way of brief introduction and to better understand the background to the inventive endeavor of the present disclosure, it is noted that the large neutral amino acid transporter 1 (LAT-1, SLC7a5) is a sodium- and pH-independent transporter, which supplies essential amino acids (e.g., leucine, phenylalanine) to cells. The functional transporter is a heterodimeric disulfide-linked complex composed of the multi-transmembrane subunit SLC7a5 and single transmembrane subunit SLC3a2 (CD98). LAT-1 is the main transporter to channel essential amino acids across such compartments such as the placenta or blood-brain barrier. In addition, LAT-1 also transports the thyroid hormones T3 and T4 (See, FRIESEMA, et al., Endocrinology, 142(10): 4339-4348 (2001)), the dopamine precursor L-DOPA, as well as amino acid-related exogenous compounds, such as the drugs melphalan and gabapentin (See, UCHINO, et a., Mol. Pharmacol 61:729-737 (2002)). Moreover, its expression is highly upregulated in several types of human cancer that are characterized by an intense demand for amino acids for metabolism and growth (See, SINGH, et. al., Int. J. Mol. Sci. 2018, 19, 1278).

Furthermore, it has been reported that the nature of the amino acid side chain influences selectivity of LAT-1 for various amino acids, with the following order in terms of increasing rate of transport: Phe>Trp>Leu>Ile>Met>His>Tyr>Val (See, KANAI, et al., J. Biol. Chem., vol. 273, No. 37, pp. 23629-23632 (1998)). However, the influence of additional boron modifications to amino acids is unknown in the art and this disclosure represents a pioneering breakthrough.

The therapeutic potential of BNCT as an effective cancer treatment rests in the selective accumulation of a sufficient amount of ¹⁰B within cancer cells.

Based on the foregoing, those of ordinary skill in the art have shown that essential amino acid transporter proteins such as LAT1 are responsible for the uptake of certain naturally occurring amino acids. See, SCALISE, et. al., Frontiers in Chem., Vol. 6, Art. 243 (June 2018). With this principle in mind, the present disclosure contemplates the synthesis of naturally occurring amino acids through borylation reactions to create Borylated Amino Acids (BAAs) with tumor seeking and tumor localizing properties for use as neutron capture agent in Boron Neutron Capture Therapy (“BNCT”) and/or Boron Proton Capture Therapy commonly known as Proton Boron Fusion Therapy (“PBFT”). See, for example, HATTORI, et. al., J. Med. Chem., 55, 6980-6984 (2012).

### (a) Amino Acid Composition(s)

In a further embodiment, a BAA with the following formula is within the scope of the of the present disclosure (“Tyrosine derivatives”):

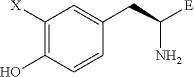

Where:
E=CO₂H, CONHB₁₂H₁₁, B(OH)₂; and
X=H, B(OH)₂, Bpin, (—O—CH₂CH₂)₂—O—B₁₂H₁₁ or BF₃⁻.

In a further embodiment, a Tyrosine derivative with the following formula is within the scope of the of the present disclosure:

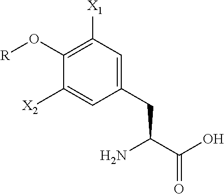

Where:
R═H, CH₃, or CF₃;
X₁═H, B(OH)₂, or BF₃⁻; and
X₂═H, B(OH)₂, or BF₃⁻

### (b) BAA Comprising Tyrosine (TC220 Hydrochloride & TC221)

Tyrosine is an essential amino acid with the following chemical formula:

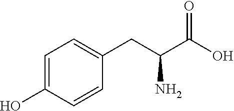 [SMILES: N[C@@H](Cc1ccc(O)cc1)C(=O)O]

and is known to readily pass the blood-brain barrier. Once in the brain, it is a precursor for the neurotransmitter's dopamine, norepinephrine, and epinephrine, better known as adrenalin. These neurotransmitters are an important part of the body's sympathetic nervous system, and their concentrations in the body and brain are directly dependent upon dietary tyrosine. Tyrosine is rapidly metabolized. Folic acid, copper, and vitamin C are cofactor nutrients of these reactions. Tyrosine is also the precursor for hormones, thyroid, catechol estrogens and the major human pigment, melanin. Tyrosine is an important amino acid in many proteins, peptides and even enkephalins, the body's natural pain reliever. Valine and other branched amino acids, and possibly tryptophan and phenylalanine may reduce tyrosine absorption. A number of genetic errors of tyrosine metabolism occur, such as Hawkins Nuria and tyrosinemia I. Most common is the increased amount of tyrosine in the blood of premature infants, which is marked by decreased motor activity, lethargy, and poor feeding. Infection and intellectual deficits may occur. Some adults also develop elevated tyrosine in their blood. This indicates a need for more vitamin C. Generally speaking, tyrosine is needed under stress, and tyrosine supplements prevent the stress-induced depletion of norepinephrine and may cure biochemical depression.

Additionally, various derivatives of tyrosine have been evaluated as tracers for whole-body imaging using PET and some are approved for specific indications, including neuroendocrine disorders and cancer. These include ¹⁸F-Fluoro-L-DOPA (DOI: 10.2967/jnumed.114.145730), ¹⁸F-Fluoro-L-alpha-methyltyrosine i.e., FAMT (INOUE, J Nucl. Med. 1998; 39 663-667 and 10.2967/jnumed.112.103069).

Furthermore, ISHIWATA, et. al. described O—[¹⁸F]fluoromethyl-L-tyrosine (e.g., ¹⁸F-FMT) and studied its biodistribution in hepatoma-bearing rats. See, Nuclear Medicine and Biology 31 (2004) 191-198. As shown, the tracer accumulated in the pancreas and there was a meaningful contrast achieved at sixty (60) minutes that afforded visualization of the tumor. However, there was some degree of defluorination noted, likely due to uptake of the tracer in the bone marrow. Defluorination of 1⁸F-FMT contrasts with no defluorination of ¹⁸F-filuoroethyltyrosine (i.e., ¹⁸F-FET) an approved imaging tracer for high-grade glioma (See, 10.2967/jnumed.114.140608 and also NCT04001257). It is noted that an elevated uptake of the foregoing PET tracers in glioma and other tumor is mediated by LAT-1, the same large neutral amino acid transporter that mediates BPA uptake into head and neck, GBM, and melanoma lesions.

Based on the foregoing, the disclosure endeavors to develop syntheses of borylated tyrosine analogs to evaluate the expression of LAT-1 in selected cell lines and show that these borylated amino acid analogs are show uptake in cancer cell lines. Furthermore, it is demonstrated in the disclosure that the tyrosine analogs are taken up by FaDu established xenografts in immunodeficient mice in a dose-dependent fashion.

Accordingly, the utilization of borylated tyrosine as a neutron capture agent in certain cancers is contemplated by the present disclosure.

In one embodiment of the present disclosure, a BAA comprising tyrosine is denoted as TC220 Hydrochloride and has the following chemical formula set forth in FIG. 1.

In one embodiment of the present disclosure, a BAA comprising tyrosine is denoted as TC221 and has the following chemical formula set forth in FIG. 2.

The synthesis of TC220 and TC221 present a challenge due to the hydroxyl of tyrosine in ortho-position to boronic acid. This hydroxyl is an electron-donating group, and it hampers the pinacol-borane deprotection step of the synthesis. As a result, known synthesis of TC220 and TC221 results in low yield and difficult-to-remove impurities. Accordingly, it is an object of the present disclosure to provide a novel synthesis of TC220 and TC221 that results in high yield and purity at up to a one (1) gram scale.

By way of background, TC220 and TC221 are very soluble in water. However, unlike BPA, the only boron carrier currently approved for use in BNCT, TC220 does not require fructose to aid solubility. Additionally, TC221 does require fructose, however, the threshold of solubility is much higher compared to BPA. As a result, using TC220 or TC221 versus BPA will allow for the administration of higher concentrations and smaller volumes of boron compounds than what is currently feasible with L-BPA fructose (or sorbitol) formulations. The clinical significance of being able to achieve higher boron concentration in a tumor will translate into higher efficacy of neutron irradiation in BNCT and/or PBFT therapy and ultimately, a lower rate of cancer recurrence.

Accordingly, it is an object of the present disclosure to teach a new and improved synthesis of TC220 and TC221.

### (c) New and Improved Synthesis of TC220 and TC221

The synthesis of TC220 (as shown in FIG. 1) and TC221 (as shown in FIG. 2) are on a laboratory scale an efficient transformation. However, the formation of L-DOPA and Tyr has been observed following the traditional Miyaura coupling (i.e., Pd coupling of bis(pinacolato)diborane followed by Na104 deprotection). The result is a major by-product and chromatography is required to remove the by-product.

In addition, the synthesis of TC220 (as shown in FIG. 1) and TC221 (as shown in FIG. 2) does not progress when the Nor C termini are deprotected unless excess BBr₃ is added. However, this presents a further problem in that the synthesis leads to rapid de-boration. It is also noted that initiating the synthesis above 0° C. also leads to de-boration.

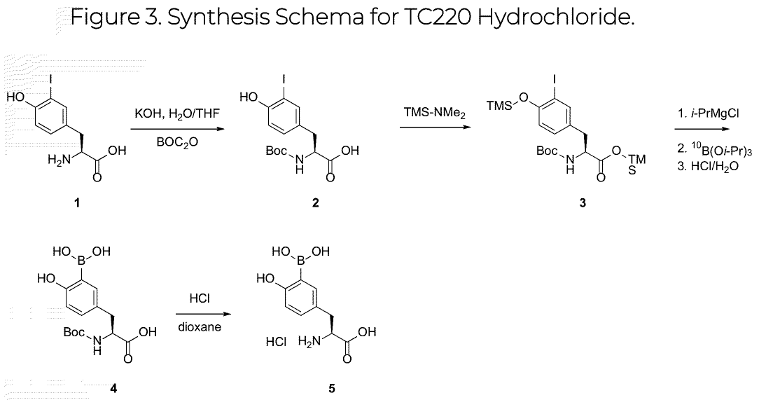

Accordingly, it is an object of the present disclosure to enable a novel improved synthesis schema for TC220 and TC221 whereby the impurity generating by Pd coupling is eliminated and the numbering of steps are reduced. (See, FIG. 3).

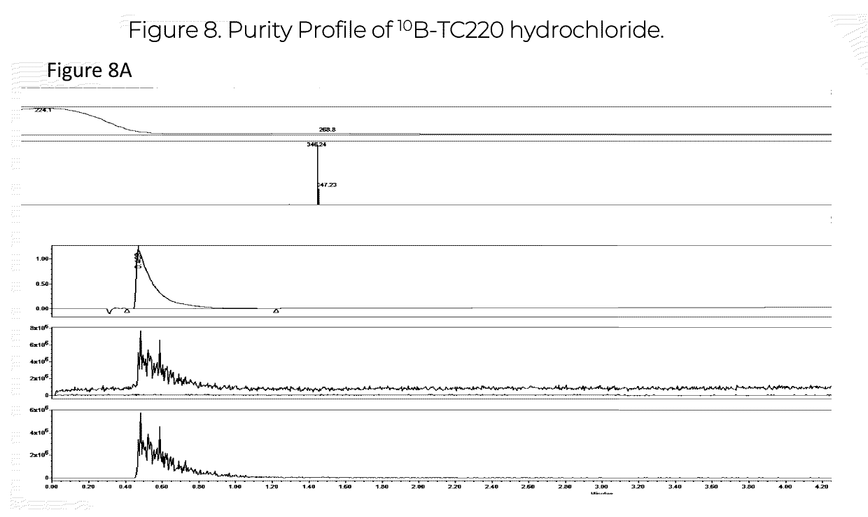

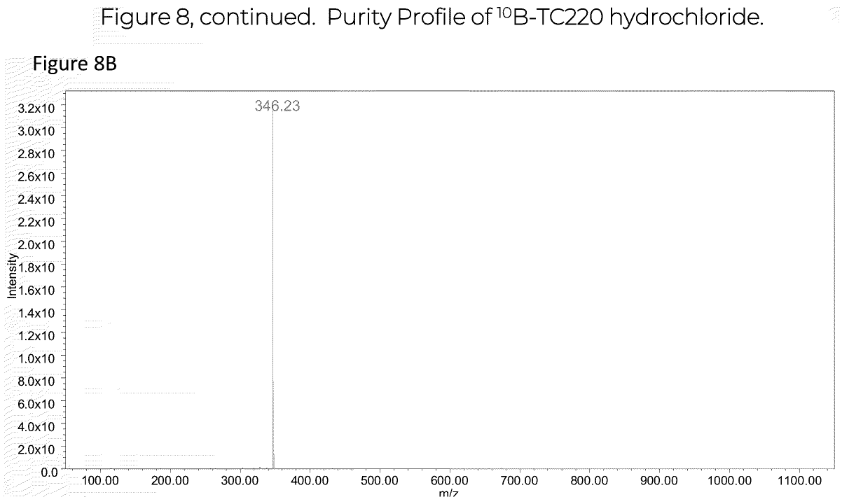

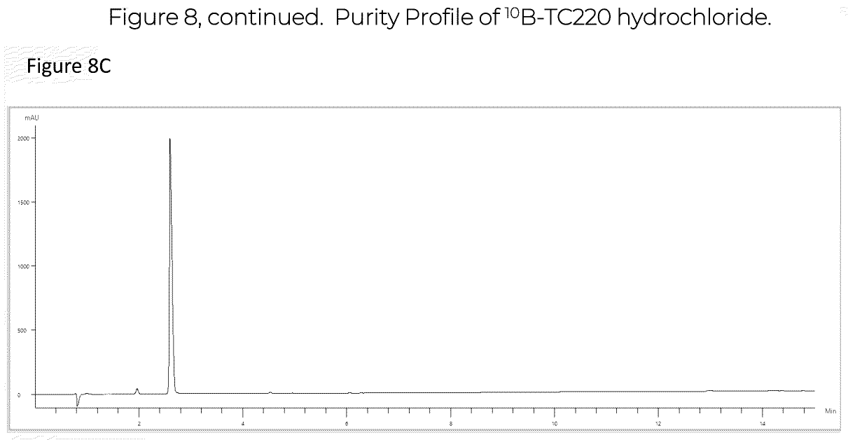

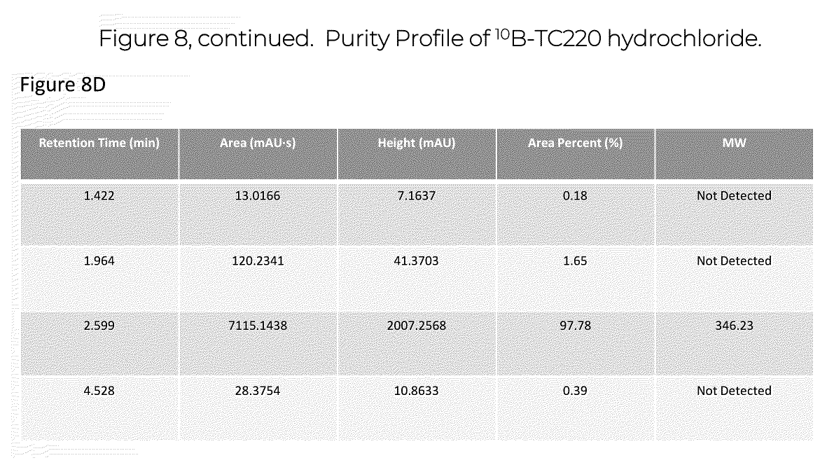

The resulting novel synthesis is prepared in such a way that no chromatography is necessary for the final purification. The purified target materials are analyzed using the using the chromatography conditions set forth in FIG. 8A and will produce TC220 (as shown in FIG. 1) and TC221 (as shown in FIG. 2) and will allow for commercial scale-up to one (1) gram.

In a preferred embodiment, the invention comprises a synthesis of TC220 and TC221 comprising the conversion of L-tyrosine to the target materials. The conversion of L-tyrosine to N-Boc-Tyr(3-Br, 4-MeO)-OMe is first presented in Ghosh, S. et al. ARKIVOC, 2009 (vii), 72-78.

The conversion of N-Boc-Tyr(3-Br, 4-MeO)-OMe to the aryl boronic acid N-Boc-Tyr(3-B(OH)₂-4-MeO)—OMe consists of a palladation followed by metal exchange to the required boronic acid. In a flame dried argon quench flask was charged with 30 mL of methanol and 12 mL of dimethoxyethane. To the solution was added 2.8 g of potassium acetate, then 5 g of Boc-Tyr(3-Br, 4-OMe)-OMe, followed by 1.3 g of tetrahydroxydiborane. Finally, to the reaction mixture is added catalytic Pd, 3 mg of Chloro[(tri-tert-butylphosphine)-2-(2-aminobiphenyl)]palladium (11). Under an argon atmosphere the reaction is stirred over night at 20° C. At the reactions completion 20 mL of water is slowly added, and allowed to quench for 30 min. The solids are removed via filtration. The organic solvents are then removed under reduced pressure. The aqueous layer is then washed three times with ethyl acetate. The organic layers are combined and concentrated under reduced pressure. The crude material is further purified via flash chromatography on silica at 25% ethyl acetate in hexanes. Upon removal of organic solvents, the target material is isolated at 75% yield as a white solid.

Following the introduction of boronic acid the synthesis diverges to form the compounds TC220 and TC221. The selective saponification of N-Boc-Tyr(3-B(OH)₂-4-MeO)—OMe to N-Boc-Tyr(3-B(OH)₂-4-MeO)—OH is highlighted in Ghosh, S. et al. ARKIVOC, 2009 (vii), 72-78. Following the saponification is the removal of the tert-butyl carbamate to reveal the structure of TC221. To a flask charged with 1 g of N-Boc-Tyr(3-B(OH)₂, 4-OMe)-OH was added a solution of 4 M hydrochloric acid in dioxane. After one hour no presence of the Boc protected starting material was observable. The volatile solvent and acid were removed under reduced pressure, and the target material was purified via preparative LC.

### VII.) Boron Neutron Cature Therapy Using TC220 & TC221

One aspect of the present disclosure is the use of TC220 and TC221 as a modality for Baron Neutron Capture Therapy (BNCT) and/or Baron Proton Capture Therapy (“BPCT”). Briefly, BNCT is a binary treatment modality in which neither component alone is lethal or toxic to the tumor. The two components comprise (i) the infusion or delivery of a capture compound, which preferentially is concentrated in the tumor, and (ii) the irradiation of the tumor site by neutrons or by protons. In BNCT, given the large cross-section of thermal neutron interactions with 10B, there is consequently a high probability of a splitting of Boron nucleus into ⁴He²⁺ and ⁷Li⁺. Given that the ionization capability of He²⁺ and Li⁺ is high, and the distances travelled are short, then the cells preferably enriched by Boron are killed and the healthy cells are damaged much less due to the lack of high concentration of boron. Given this, the advantage of BNCT is the destruction of tumor cells without a highly traumatic surgical procedure. However, as will be understood by one of skill in the art, success is predicated high concentration and selective localization of ¹⁰B in tumor cells.

In one embodiment, ¹⁰B is concentrated on TC220 and/or TC221. The TC220 and/or TC221 are then given to a patient and the TC220 & TC221 is localized into a tumor cell. The TC220 & TC221 containing ¹⁰B are concentrated into the tumor and the tumor is irradiated using epithermal neutrons.

The tumor cells are destroyed.

### VIII. Proton Boron Fusion Therapy Using BAAs

Another aspect of the present disclosure is the use of TC220 & TC221 as a modality for Proton Boron Fusion Therapy (PBFT). Briefly, the proton boron fusion reaction was introduced in the 1960s. Three alpha particles are emitted after the reaction between a proton (¹H) and a boron particle (¹¹B). These three alpha particles provide the damage to the tumor cell, just as in the case of alpha particles in BNCT. Theoretically, in the case of PBFT, the therapy efficacy per incident particle is three times (3×) greater than that of BNCT. In addition, because the proton beam has the advantage of a Bragg-peak characteristic, normal tissue damage can be reduced. Generally speaking, many studies for tumor treatment using alpha particles have been performed. In order to take advantage of alpha particles for dose delivery, two key points should be considered. First, the boron uptake should be labeled accurately to the target cell. As mentioned previously, alpha particles are generated where the boronate compound is accumulated. If this happens in normal tissue near the tumor region, alpha particles will damage the normal tissue as well as the tumor cell. Second, the number of generated alpha particles is also a significant factor for effective therapy. By using PBFT, a more effective therapy can be realized compared to BNCT or conventional proton therapy alone.

In one embodiment, ¹⁰B and/or ¹¹B is concentrated on a TC220 & TC221. The TC220 & TC221 is then given to a patient and the TC220 & TC221 is localized into a tumor cell. The TC220 & TC221 containing ¹⁰B and/or ¹¹B are concentrated into the tumor and the tumor is irradiated using epithermal neutrons. The tumor cells are destroyed.

### IX. Methods of Delivering TC220 & TC221 to a Cell

As will be appreciated by one of ordinary skill in the art, the ability to efficiently deliver high concentrations of Boron to a cell is an advantage of the present invention.

It is shown that the TC220 & TC221 of the present disclosure enables a higher amount of boron to be administered to a cell safely in mammals. Briefly, TC220 & TC221 of the disclosure are prepared as set forth in the disclosure. The resulting TC220 & TC221 are taken up by the tumor cell by the upregulated LAT-1 transporter protein.

### X.) Kits/Articles of Manufacture

For use in the laboratory, prognostic, prophylactic, diagnostic, and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise a TC220 & TC221 or several TC220(s) & TC221(s) of the disclosure. Kits can comprise a container comprising a drug unit. The kit can include all or part of the TC220(s) & TC221(s) and/or diagnostic assays for detecting cancer and/or other immunological disorders.

The kit of the invention will typically comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic, or laboratory application, and can also indicate directions for either in vivo or in vitro use, such as those described herein. Directions and or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing, or prognosing a condition, such as a cancer or other immunological disorder.

The terms “kit” and “article of manufacture” can be used as synonyms.

In another embodiment of the invention, an article(s) of manufacture containing compositions, such as TC220(s) & TC221(s) of the disclosure. The article of manufacture typically comprises at least one container and at least one label. Suitable containers include, for example, bottles, vials, syringes, and test tubes. The containers can be formed from a variety of materials such as glass, metal, or plastic. The container can hold one or several TC220(s) & TC221(s) and/or one or more therapeutics doses of TC220(s) & TC221(s).

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be a TC220 & TC221 of the present disclosure.

The article of manufacture can further comprise a second container comprising a pharmaceutically acceptable buffer, such as phosphate-buffered saline, Ringer's solution, and/or dextrose solution. It can further include other materials desirable from a commercial and user standpoint, including other buffers, diluents, filters, stirrers, needles, syringes, and/or package inserts with indications and/or instructions for use.

### Exemplary Embodiments

1) A composition comprising a chemical structure as follows:

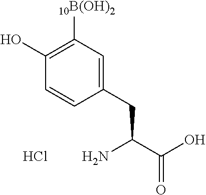

2) A composition comprising a chemical structure as follows:

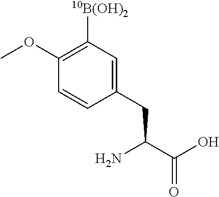

3) A composition produced by a method of conversion of L-tyrosine to a target material, comprising the following synthesis:

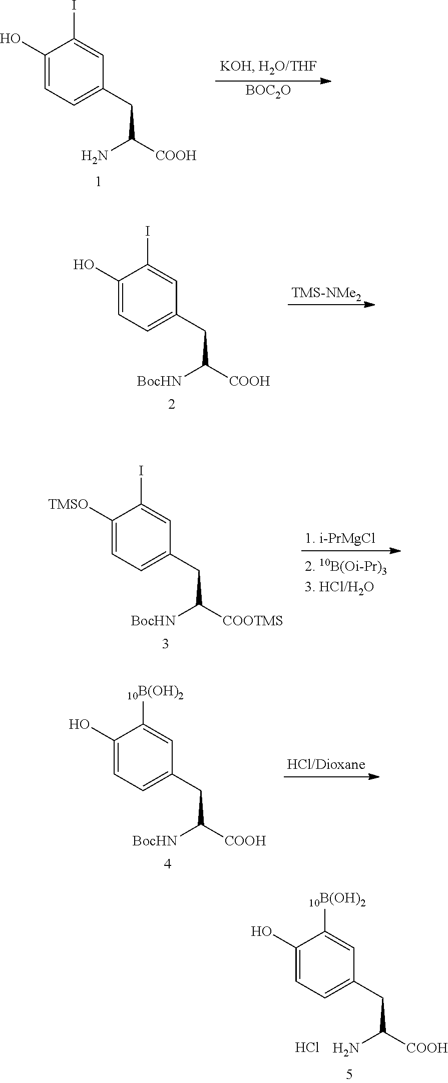

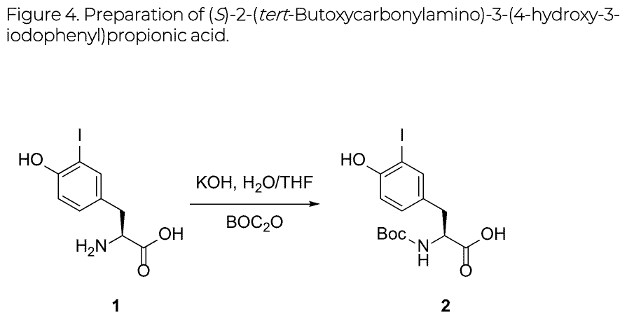

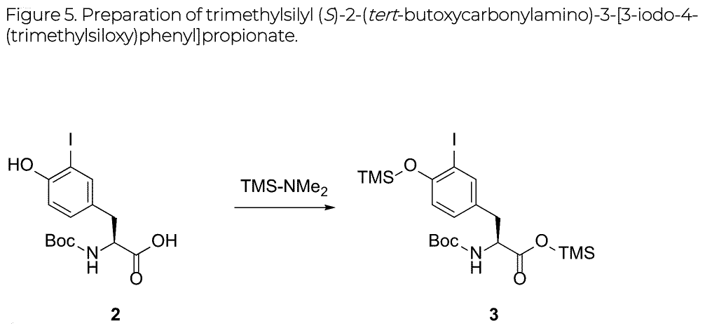

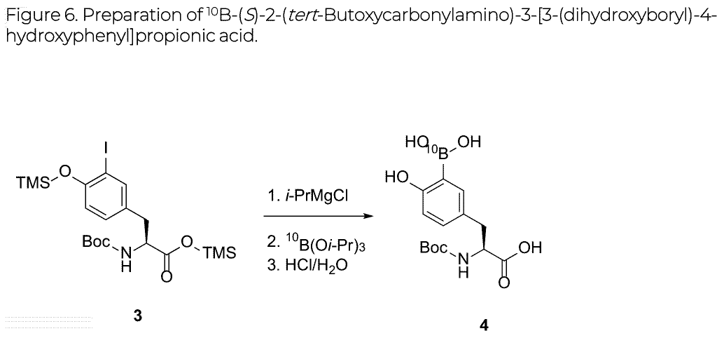

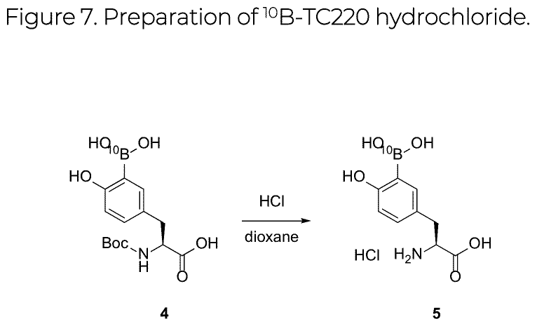

4) The method of claim 3, wherein the composition that is produced is set forth in FIG. 1.
5) A method comprising the synthesis of (S)-2-(tert-Butoxycarbonylamino)-3-(4-hydroxy-3-iodophenyl)propionic acid as shown in FIG. 4.
6) The method of claim 5, further comprising the synthesis of trimethylsilyl (S)-2-(tert-butoxycarbonylamino)-3-[3-iodo-(trimethylsiloxy)phenyl]propionate as shown in FIG. 5.
7) The method of claim 6, further comprising the synthesis of ¹B—(S)-2-(tert-Butoxycarbonylamino)-3-[3-(dihydroxyboryl)-4-hydroxyphenyl]propionic acid as shown in FIG. 6.
8) The method of claim 7, further comprising the synthesis of target material (4) into target material (5) as shown in FIG. 7.
9) A composition produced by a method of conversion of L-tyrosine to a target material, comprising the following synthesis:

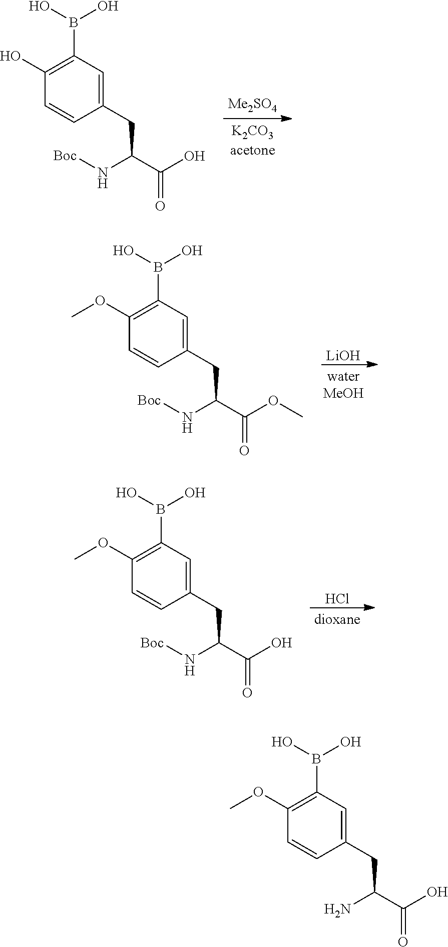

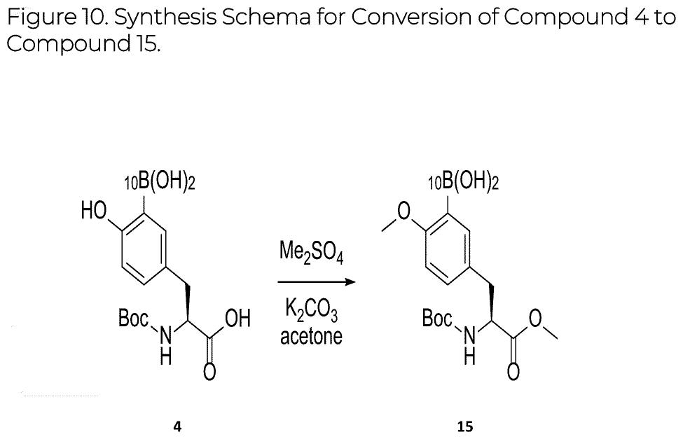

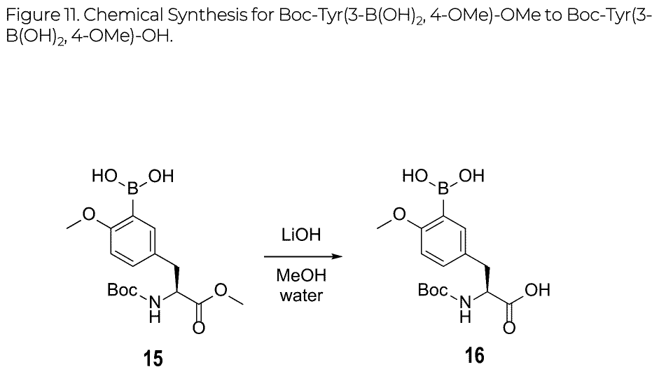

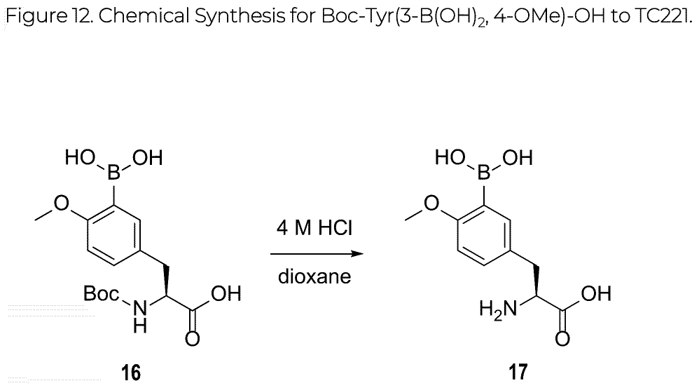

10) The method of claim 9, wherein the composition that is produced is set forth in FIG. 2.
11) A method comprising the synthesis for Conversion of ¹⁰B—(S)-2-(tert-Butoxycarbonylamino)-3-[3-(dihydroxyboryl)-4-hydroxyphenyl]propionic acid to Methyl ¹⁰B—(S)-2-(tert-Butoxycarbonylamino)-3-[3-(dihydroxyboryl)-4-hydroxyphenyl]propionate as shown in FIG. 10.
12) The method of claim 11, further comprising the synthesis for Boc-Tyr(3-B(OH)₂, 4-OMe)-OMe to Boc-Tyr(3-B(OH)₂, 4-OMe)-OH as shown in FIG. 11.
13) The method of claim 12, further comprising the synthesis of target material (16) into target material (17) as shown in FIG. 12.
14) A kit comprising the composition of claim 1.
15) A kit comprising the composition of claim 2.
16) A Dosage Unit Form comprising a composition of claim 1.
17) A Dosage Unit Form comprising a composition of claim 2.
18) The Human Unit Form of claim 16, wherein the Human Unit Form is used in Baron Neutron Capture Therapy (BNCT).
19) The Human Unit Form of claim 17, wherein the Human Unit Form is used in Baron Neutron Capture Therapy (BNCT).
20) The Human Unit Form of claim 16, wherein the Human Unit Form is used in Proton Boron Fusion Therapy (PBFT).
21) The Human Unit Form of claim 17, wherein the Human Unit Form is used in Proton Boron Fusion Therapy (PBFT).

### EXAMPLES

Various aspects of the invention are further described and illustrated by way of the several examples that follow, none of which is intended to limit the scope of the invention.

### Example 1: Preparation of TC220 Hydrochloride

The overall synthesis schema for producing TC220 Hydrochloride (FIG. 1) is shown in FIG. 3 and is performed using chemical methods.

### Example 2: Preparation of (S)-2-(tert-Butoxycarbonylamino)-3-4-hydroxy-3-iodophenytoropionic acid

Using the starting material (Chemical Structure No. 1) as shown in FIG. 4, the following synthesis was performed using the following methods.

Briefly, A 3-L three-neck round bottom flask equipped with a mechanical stirrer and thermometer was charged with water (760 mL) and potassium hydroxide (63.1 g, assay 86.7%, 0.977 mol). The mixture was stirred until potassium hydroxide dissolved, and 3-iodo-L-tyrosine (1) (150 g, 0.489 mol) was added. The reaction was cooled to 9° C., and a solution of di-tert-butyl dicarbonate (107 g, 0.491 mmol) in THE (150 mL) was added. The cooling was then removed, and the mixture stirred at room temperature for 18 hours.

Then, the reaction mixture was placed on a rotary evaporator and concentrated under reduced pressure at 45° C. until its might reached 700 g. The residue was then transferred into a 5-L three-neck round-bottom flask equipped with a mechanical stirrer and pH meter. MTBE (700 mL) was added.

The mixture was acidified to pH 3.1 by carefully adding citric acid hydrate in 20-g portions (the total of 240 g of citric acid hydrate was added). As observed: the mixture foamed.

Subsequently, Heptane (350 mL) was added, and the upper organic layer was separated. The organic layer was separated, extracted twice with water (700 mL), and stirred with sodium sulfate (100 g) for 30 min. The suspension was filtered, and the filter cake was washed with MTBE (2×50 mL). The filtrate concentrated on a rotary evaporator in a 5-L flask under reduced pressure at 30° C. until most of the organic solvents evaporated. Heptane (200 mL) was added, and concentration continued until the mixture foamed and filled most of the flask. More heptane (200 mL) was added, and the mixture was again evaporated until organic solvents stopped distilling. The residue was placed under vacuum and kept for 3 days at room temperature. This afforded the title compound (Chemical Structure No. 2) (198 g, 99%) as an off-white solid foam. Confirmatory NMR was performed using standard methods and resulted in the following: ¹H NMR (500 MHz, CD₃OD) 7.57 (d, J=2.1 Hz, 1H), 7.06 (dd, J=8.3, 2.1 Hz, 1H), 6.77 (d, J=8.2 Hz, 1H), 4.28 (dd, J=8.9, 5.0 Hz, 1H), 3.06 (dd, J=14.0, 5.1 Hz, 1H), 2.79 (dd, J=14.0, 9.0 Hz, 1H), 1.42 (s, 9H). (See, FIG. 4).

### Examle 3: Preparation of trimethylsilyl (S)-2-tert-butoxycarbonylamino)-3-[3-iodo-4-(trimethylsiloxy)phenyl]propionate

Using the starting material (Chemical Structure No. 2) as shown in FIG. 5, the following synthesis was performed using the following methods.

Briefly, A 500-mL three-neck round bottom flask equipped with a magnetic stirrer, thermoregulator, reflux condenser, and a nitrogen inlet over the condenser was charged with N,N-dimethyltrimethylsilylamine (117 mL, 0.732 mol). Chemical Structure No. 2 (60.0 g, 0.147 mol) was then added in portions in a flow of nitrogen. Notably, it is observed that foaming and gas evolution is possible. The internal temperature of the mixture increased to 40° C. during the addition. The mixture was then heated at reflux at 94-96° C. for 18 h. Post-heating, the temperature was reduced to 20° C., after which the mixture was rapidly transferred into a 500-mL single-neck round-bottom flask. The excess reagent was then evaporated on a rotary evaporator under reduced pressure at 70° C. for 1 h. Upon completion of this interval, the flask was cooled back to 20° C. in a vacuum before being filled with nitrogen. Title compound (Chemical Structure No. 3) was obtained as a brown oil in 94% yield (76.5 g). (See, FIG. 5).

### Example 4: Preparation of ¹⁰B—(S)-2-(tert-Butoxycarbonylamino)-3-[3-(dihydroxyboryl)-4-hydroxyphenyl]propionic acid

Using the starting material (Chemical Structure No. 3) as shown in FIG. 6, the following synthesis was performed using the following methods.

Briefly, Chemical Structure No. 3 (76.5 g, 139 mmol) was first dissolved in anhydrous THE (320 mL) under a nitrogen atmosphere. This solution was subsequently transferred via a cannula into a nitrogen-prefilled 2-L three-neck flask, equipped with a magnetic stirrer, addition funnel, nitrogen inlet, and thermometer. The flask contents were cooled to −25° C. before adding a 2M solution of isopropylmagnesium chloride in THE (175 mL) via the addition funnel over a 30-minute period, maintaining an internal temperature of −27 to −23° C. The funnel was rinsed with anhydrous THE (15 mL) which was also added to the mixture. The reaction proceeded for 1 hour at −27 to −23° C. At this point, the mixture was cooled to −50° C., and ¹⁰B-triisopropyl borate (65 mL, 283 mmol) was added via the addition funnel over a 10 min period, at −55-−50° C. Following the addition, the mixture was permitted to gradually warm to 7° C. over a period of 2 h.

Then, the reaction was further cooled to maintain an internal temperature below 10° C. and quenched by the addition of a mixture of 2M aqueous hydrochloric acid (170 mL) and water (140 mL). The reaction pH was adjusted to 3 using 1M aqueous sodium hydroxide. The organic phase was separated and transferred into a 500-mL three-neck flask equipped with a magnetic stirrer, pH-meter, addition funnel, and nitrogen inlet. Then, 1M aqueous sodium hydroxide was added (approximately 175 mL) under nitrogen until the mixture's pH reached 6.85. The resultant mixture was subsequently concentrated using a rotary evaporator under reduced pressure, without heating, for 4 h until the residue weighed 270 g.

Then, this mixture was extracted twice under nitrogen using MTBE (280 ml, then 250 mL). The aqueous layer was isolated and transferred into a 1-L three-neck flask, equipped with a magnetic stirrer, pH-meter, addition funnel, and nitrogen inlet. MTBE (300 mL) was added to the aqueous layer, followed by the dropwise addition of concentrated 37% hydrochloric acid (approximately 16 g) under nitrogen to achieve a pH of 3.1. The organic layer was promptly separated and stirred with magnesium sulfate (26 g) for 10 minutes under a nitrogen atmosphere. This suspension was quickly filtered, and the filter cake rinsed with MTBE (2×25 mL). The filtrate was evaporated using a rotary evaporator under reduced pressure without heating and subsequently kept under vacuum for 18 h to yield crude Chemical Structure No. 4 as an off-white foam (39.5 g).

Finally, this foam was stirred under nitrogen with ethyl acetate (550 mL) for 4 h. The resulting suspension was then filtered through a 10-μm pad filter, and the filter cake was dried under vacuum for 4 d. The resultant solid was re-treated with ethyl acetate as outlined above, yielding the pure title Compound (Chemical Structure No. 4) as an off-white solid (20.1 g, 45% yield). Confirmatory NMR was performed using standard methods and resulted in the following: ¹H NMR (500 MHz, CD₃OD) 7.65-7.0 (br m, 2H), 6.77 (d, J=8.3 Hz, 1H), 4.27 (dd, J=8.5, 5.0 Hz, 1H), 3.05 (dd, J=13.8, 5.1 Hz, 1H), 2.83 (dd, J=13.9, 8.7 Hz, 1H), 1.38 (s, 9H). (See, FIG. 6).

### Example 5: Preparation of ¹⁰B-TC220 Hydrochloride

Using the starting material (Chemical Structure No. 4) as shown in FIG. 7, the following synthesis was performed using the following methods.

Briefly, A 1-L three-neck flask, equipped with a magnetic stirrer and nitrogen inlet, was charged with dioxane (250 mL) and a 4M solution of hydrogen chloride in dioxane (250 mL). Under a nitrogen atmosphere, Chemical Structure No. 4 (24.7 g, 76.2 mmol) was added portion wise. The mixture was subsequently stirred at ambient temperature for a duration of 6 h, then filtered through a 10 μm pad filter. The resulting filter cake was left to dry under vacuum at room temperature overnight.

Then, the dry cake was then dissolved in water (140 mL), and the filtrate was clarified by passage through a 2.5 μm filter. This was followed by lyophilization to afford ¹⁰B-TC220 hydrochloride (Chemical Structure No. 5) (See also, FIG. 1) as a white solid, with an 86% yield (17.0 g). Confirmatory NMR was performed using standard methods and resulted in the following: ¹H NMR (500 MHz, D₂O) 7.36 (d, J=2.4 Hz, 1H), 7.16 (dd, J=8.4, 2.4 Hz, 1H), 6.77 (d, J=8.3 Hz, 1H), 4.13 (dd, J=7.7, 5.5 Hz, 1H), 3.15 (dd, J=14.8, 5.5 Hz, 1H), 3.03 (dd, J=14.8, 7.7 Hz, 1H). (See, FIG. 7).

### Example 6: Purity Analysis of ¹⁰B-TC220 Hydrochloride

A purity analysis of TC220 was performed using standard methods in the art. Briefly, UPLC/MS was performed using a C18 column with a gradient from 2% to 20% B. The solvent conditions where 0.1% formic acid in water (A), and 0.1% formic acid in acetonitrile (B), at 0.500 ml/min flow rate. Maximum absorbance was found at 225 nm.

### Example 7: Synthesis Schema for Conversion of TC220 Hydrochloride to TC221

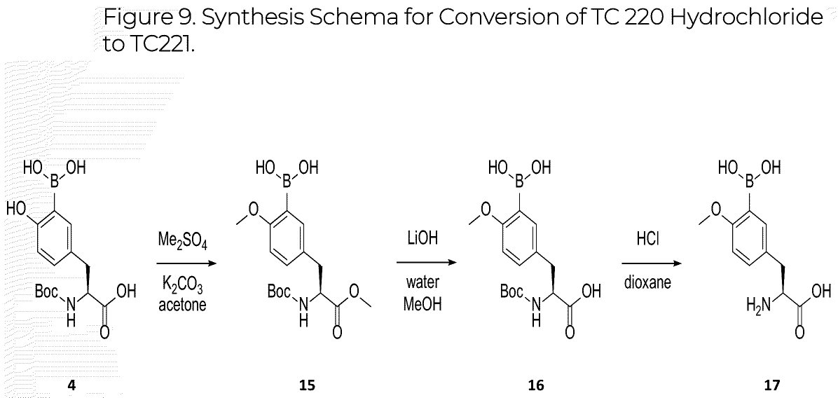

The overall synthesis schema for converting TC220 Hydrochloride (FIG. 1) to TC221 (FIG. 2) is shown in FIG. 9 and is performed using chemical methods.

### Example 8: Synthesis Schema for Conversion of ¹⁰B—(S)-2-(tert-Butoxycarbonylamino)-3-[3-(dihydroxyboryl)-4-hydroxyohenyl]propionic acid to Methyl ¹⁰B—(S)-2-(tert-Butoxycarbonylamino)-3-[3-(dihydroxyboryl)-4-hydroxyphenyl]propionate

Using the starting material (Chemical Structure No. 4) as shown in FIG. 9, the following synthesis was performed using the following methods.

Briefly, to a solution of Chemical Structure No. 4 (5.0 g, 17.84 mmol) in acetone (50 mL) was added K₂CO₃(7.4 g, 53.51 mmol) and Me₂SO₄ (2.5 g, 44.6 mmol) at room temperature. The reaction mixture was stirred at same temperature for 12 h. The reaction mixture was filtered through pad of Celite and washed with acetone (50 mL). The acetone layer was concentrated under vacuum to give Chemical Structure No. 15 as an off-white tacky material. (See, FIG. 10).

### Example 9: Preparation of (S)₂-(tert-butoxycarbonylamino)-3-4-methoxy-3-boronic acid)propionic acid

The synthesis of N-Boc-Tyr(3-B(OH)₂, 4-OMe)-OMe to N-Boc-Tyr(3-B(OH)₂, 4-OMe)-OH is shown in FIG. 11 and is performed using standard methods. See, GHOSH, et. al., Arkivoc (2009) (vii) pp. 72-78. The modification does not utilize a procedure for demethylation with BBr₃ and is shown in FIG. 9, so as to maintain the methyl ether.

### Example 10: Preparation of (S)-2-(amino)-3-(4-methoxy-3-boronic acid)propionic acid. TC221

The synthesis of N-Boc-Tyr(3-B(OH)₂, 4-OMe)-OH to TC221 is shown in FIG. 12 and utilizes a further synthesis modification that is within the scope of this disclosure. Briefly, to a flask charged with 1.0 g of N-Boc-Tyr(3-B(OH)₂, 4-Ome)-OH was added a solution of 4 M hydrochloric acid in dioxane. After one (1) hr. no presence of the Boc protected starting material was observable. The volatile solvent and acid were removed under reduced pressure, and the target material was purified via preparative LC using the protocol. Briefly, a C18 preparative column was used with dimensions of 30 mm×100 mm. The flow was set at 50 mL/min. The gradient used was set forth in the following 0% acetonitrile to 20% acetonitrile in water over 20 minutes.

The resulting TC221 has the structure set forth in FIG. 2.

### Example 11: Human Clinical Trials for the Treatment of Human Carcinomas Through the Use of TC220 & TC221

TC220 and/or TC221 are synthesized in accordance with the present invention which specifically accumulate in a tumor cell and are used in the treatment of certain tumors and other immunological disorders and/or other diseases. In connection with each of these indications, two clinical approaches are successfully pursued.

I.) Adjunctive therapy: In adjunctive therapy, patients are treated with TC220 and/or TC221 in combination with a chemotherapeutic or pharmaceutical or biopharmaceutical agent or a combination thereof. Primary cancer targets are treated under standard protocols by the addition of TC220 and/or TC221 and then irradiated. Protocol designs address effectiveness as assessed by the following examples, including but not limited to, reduction in tumor mass of primary or metastatic lesions, increased progression free survival, overall survival, improvement of patients' health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.

II.) Monotherapy: In connection with the use of the TC220 and/or TC221 in monotherapy of tumors, the TC220 and/or TC221 are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patients' health, disease stabilization, as well as the ability to reduce usual doses of standard chemotherapy and other biologic agents.

### Dosage

Dosage regimens may be adjusted to provide the optimum desired response. For example, a single TC220 and/or TC221 injection may be administered, several divided doses may be administered over time, or the dose may be proportionally reduced or increased as indicated by the exigencies of the therapeutic situation. “Dosage Unit Form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the TC220 and/or TC221, the individual mechanics of the irradiation mechanism (reactor) and the particular therapeutic or prophylactic effect to be achieved, and (b) the limitations inherent in the art of compounding such a compound for the treatment of sensitivity in individuals.

### Clinical Development Plan (CDP)

The CDP follows and develops treatments of cancer(s) and/or immunological disorders using TC220 and/or TC221 of the disclosure which are then irradiated using Neutron Capture Therapy in connection with adjunctive therapy or monotherapy. Trials initially demonstrate safety and thereafter confirm efficacy in repeat doses. Trials are open label comparing standard chemotherapy with standard therapy plus TC220 and/or TC221 which are then irradiated using Boron Neutron Capture Therapy. As will be appreciated, one non-limiting criteria that can be utilized in connection with enrollment of patients is concentration of TC220 and/or TC221 in a tumor as determined by standard detection methods known in the art.

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Naturally Occuring Amino Acids.

| SINGLE | THREE | |
|---|---|---|
| LETTER | LETTER | FULL NAME |
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

1. A composition produced by a process of conversion of L-tyrosine to a target material, comprising the following synthesis:

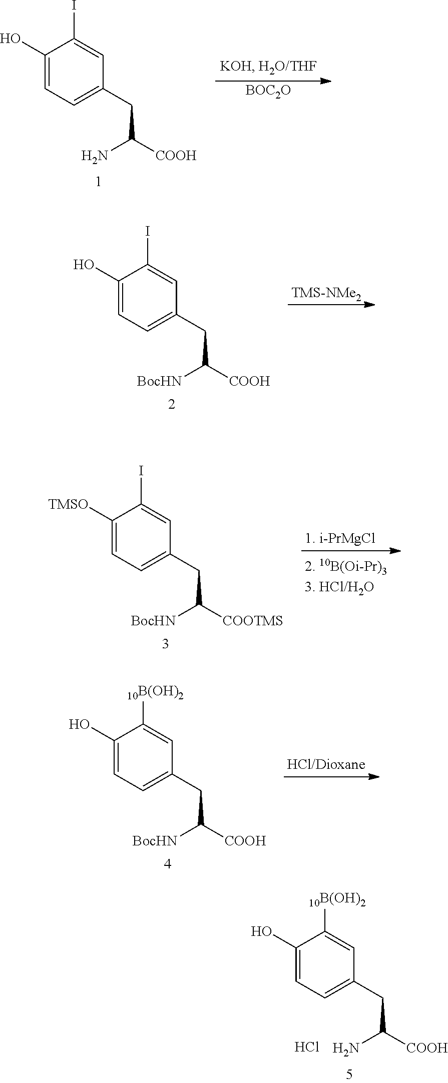

2. A composition produced by a process of conversion of L-tyrosine to a target material, comprising the following synthesis:

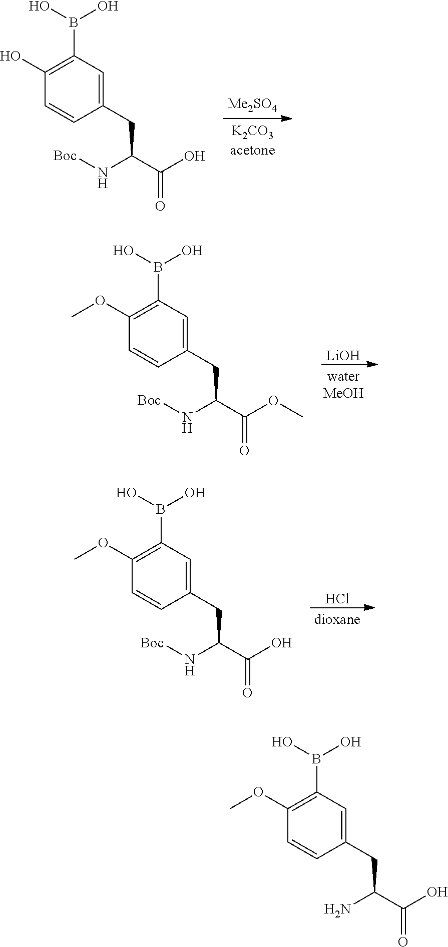

3. The process of claim 1, wherein the composition that is produced is denoted TC220 and is set forth in FIG. 1.

4. The process of claim 2, wherein the composition that is produced is denoted TC221 and is set forth in FIG. 2.

5. A kit comprising the composition produced by the process of claim 1.

6. A kit comprising the composition produced by the process of claim 2.

7. A Dosage Unit Form comprising a composition produced by the process of claim 1.

8. A Dosage Unit Form comprising a composition produced by the process of claim 2.

9. The Dosage Unit Form of claim 7, wherein the Dosage Unit Form is used in Boron Neutron Capture Therapy (BNCT).

10. The Dosage Unit Form of claim 8, wherein the Dosage Unit Form is used in Boron Neutron Capture Therapy (BNCT).

11. The Dosage Unit Form of claim 7, wherein the Dosage Unit Form is used in Proton Boron Fusion Therapy (PBFT).

12. The Dosage Unit Form of claim 8, wherein the Dosage Unit Form is used in Proton Boron Fusion Therapy (PBFT).
